(19) United States
(12) Patent Application Publication
(10) Pub. No.: US 2011/0288045 A1
(43) Pub. Date: Nov. 24, 2011

(54) WET GRANULATION OF TENOFOVIR, EMTRICITABINE AND EFAVIRENZ

(75) Inventors: Rita Ramos, Amsterdam (NL); Evanthia Dova, Amsterdam (NL); Samir Kulkarni, Amsterdam (NL); Marcel Hoffmann, Amsterdam (NL)

(73) Assignee: ULTIMORPHIX TECHNOLOGIES B.V., Amsterdam (NL)

(21) Appl. No.: 13/129,676

(22) PCT Filed: Nov. 19, 2009
(86) PCT No.: PCT/NL09/00227

§ 371 Date: Aug. 11, 2011

Related U.S. Application Data
(60) Provisional application No. 61/116,925, filed on Nov. 21, 2008.
(60) Provisional application No. 61/179,146, filed on May 18, 2009.

(51) Int. Cl. A61K 31/7068; A61P 31/18; A61P 31/12

(52) U.S. Cl. 514/49

(57) ABSTRACT

The present invention describes a method and composition for a pharmaceutical product based on Tenofovir disoproxil hemifumarate, Emtricitabine and Efavirenz. The composition can be prepared by a process comprising a wet granulation step to produce a stable dosage form suitable for the treatment of HIV in essential absence of known degradation products.

### CROSS-REFERENCE TO RELATED APPLICATIONS

This application is the National Stage of International Application No. PCT/NL2009/000227, filed Nov. 19, 2009, which claims the benefit of U.S. Provisional Application Nos. 61/116,925, filed Nov. 21, 2008, and 61/179,146, filed May 18, 2009, the contents of all of which are incorporated by reference herein.

### FIELD OF THE INVENTION

This application relates to products for the treatment of viral infections, in particular HIV infections, using the known antiviral compounds Efavirenz (tradename Sustiva, also known as EFV), Emtricitabine (tradename Emtriva, also known as FTC) and Tenofovir DF (disoproxil fumarate, also known as TDF) (tradename Viread, sold in combination with Emtricitabine under the tradename Truvada).

### BACKGROUND OF THE INVENTION

The Truvada product is produced by wet granulation of Emtricitabine and Tenofovir DF (WO 04/64845), which under the circumstances produces a chemically stable dosage form. This product does not contain Efavirenz.

HIV therapy using Efavirenz as well as Emtricitabine and Tenofovir DF has been considered desirable (hereafter “triple combination”; see WO 04/64845). Manufacturing a commercially viable triple combination product, however, would require that the final product meet stringent FDA requirements for bioequivalence to the commercial products, Viread (Tenofovir disoproxil fumarate), Emtriva (Emtricitabine), Sustiva (Efavirenz), and that the tablet be of suitable size for patients to easily swallow.

US2007099902A1 describes the formulation of a so-called triple product of Efavirenz, Tenofovir Disoproxil Fumarate (Tenofovir DF) and Emtricitabine (see WO046485 for the triple product). In the development of this triple product several obstacles had to be overcome. Initial attempts by manufacturing the three drugs into a unitary formulation, essentially homogenous composition via conventional wet granulation did not yield the desired chemically stable tablet. It was found that the Tenofovir DF degraded rapidly and the Efavirenz formulation was incompatible with Tenofovir, later on this effect was associated with the surfactant (sodium lauryl sulphate) used in the Efavirenz formulation. The dry granulation of the three components in absence of the surfactant was also not successful in terms of the desired bioequivalence as cMax and AUC were below the desired level. Combination tablets were attempted by wet granulation of Efavirenz combined with dry granulated Truvada products (Tenofovir DF and Emtricitabine) and pressing the two granulates together. This also failed to produce the desired bioequivalence.

EP1890681 describes the attempts to wet granulate the three API's together. Due to the low solubility of Efavirenz necessitating the use of sufficient water apparently led to a formulation in which the Tenofovir DF was unstable, thereby forming a eutectic mixture with Emtricitabine that, after granulation and drying was of a glassy or amorphous nature. The use of excessive amounts of recipients to counter this led to a dosage form which would be too large. Thus Tenofovir DF and Emtricitabine were dry formulated, in the essential absence of water to provide a stable product.

US2007099902 described the achievement of the desired stability and bioequivalence of the Sustiva (triple) product by formulating a two component dosage from. The first component comprising Tenofovir DF and Emtricitabine and the second component comprising Efavirenz and a surfactant. Any surfactant present in this formulation is not in stabilizing contact with Tenofovir DF.

So it is still not possible to produce a triple formulation that meets all the above criteria and that can be produced at an economic scale to further reduce the cost, for instance for countries that have a low income per head and that have a profound need of large volumes of HIV drugs.

The chemical stability of active ingredients in a pharmaceutical formulation is of concern to minimize the generation of impurities and ensure adequate shelf life. Thus, there clearly exists a need to devise a suitable manufacturing process for the formulation of dosage forms containing Emtricitabine in combination with other antiretroviral agents such as Tenofovir and/or Efavirenz so that the formulation remains stable during the entire shelf life with minimum levels of degradation products.

Recently a Tenofovir disoproxil hemifumarate (or co-crystal) has been described, TDFA 2:1 in WO2008143500.

### SUMMARY OF THE INVENTION

The present inventors have now found that this hemifumarate is capable of being wet granulated together with Efavirenz and Emtricitabine without any of the above observed adverse effects.

Thus in a first aspect the invention relates to a composition comprising Tenofovir disoproxil hemifumarate, Emtricitabine and Efavirenz. The composition of the invention does not need to be formulated in so-called multicomponent formulations such as disclosed in US2007099902. The Tenofovir disoproxil hemifumarate, Efavirenz and Emtricitabine are in an essentially homogenous composition, meaning that they are in direct physical contact with each other, without the formation degradation products of the type such as disclosed in US2007099902. US 2007099902 discloses several types of degradation products that may be present in varying amounts. For instance, degradation products of TDF are certain (mixed) dimeric degradation products, mono-POC PMPA, and/or FTU. The present compositions are essentially free from degradation products, in particular free from degradation products having a Mw of about 935 (mixed dimer) and/or 1050 dimer. Preferable the composition is free of pharmaceutically unacceptable amounts of these degradation products.

A “pharmaceutically unacceptable amount” is defined as the following amounts of each degradation product. Degradation products optionally are assayed in either an absolute or incremental amount. The absolute or total amount of degradation product is simply the amount found in the test article. The incremental amount is the additional amount of degradation product appearing in the product over that which was present (if any) in the API starting material. Moreover, the amount of degradation product optionally is measured at least two points in time. One is at the moment of preparation. One is at the time of release into the marketplace. Another is after exposure to storage conditions under the conditions described below, i.e., the shelf life as set forth below.

Total Amount at preparation: No more than about 3%, ordinarily about 1.5%, of mono-POC PMPA, No more than about 1%, ordinarily about 0.5% of Dimer, No more than about 0.5%, ordinarily about 0.25% of Mixed Dimer. Less than about 0.5%, ordinarily about 0.2% of FTU.

Total Amounts at Release (First Commercial Sale): No more than about 3%, ordinarily about 1.5%, of mono-POC PMPA, No more than about 1%, ordinarily about 0.5% of Dimer, No more than about 0.5%, ordinarily about 0.25% of Mixed Dimer. Less than about 0.5%, ordinarily about 0.2% of FTU.

Total Amounts at Shelf Life (Storage at 25[deg.] C./60% RH for 24 mo.) No more than about 10%, ordinarily about 5% of mono-POC PMPA, No more than about 2%, ordinarily about 1% of Dimer, No more than about 2%, ordinarily about 1% of Mixed Dimer. No more than about 4%, ordinarily about 2% of FTU

Incremental Amounts at Release (First Commercial Sale) No more than about 2%, ordinarily about 0.5%, of mono-POC PMPA, No more than about 0.6%, ordinarily about 0.1% of Dimer, No more than about 0.3%, ordinarily about 0.05% of Mixed Dimer. Less than about 0.4%, ordinarily about 0.1% of FTU Incremental Amounts at Shelf Life (Storage at 25[deg.] C./60% RH for 24 mo.) No more than about 9%, ordinarily about 4% of mono-POC PMPA, No more than about 1.6%, ordinarily about 0.6% of Dimer, No more than about 1.8%, ordinarily about 0.8% of Mixed Dimer. No more than about 3.9%, ordinarily about 1.9% of FTU.

The percentage of degradation products is the amount of degradation product as measured by HPLC retention time comparison. In the HPLC retention time comparison, the retention time of the main peaks observed in the tablets is required to be within 2% of the retention time of the main peaks in the a reference standard preparation containing Efavirenz, Emtricitabine, and Tenofovir DF in an assay which has been shown to be specific for Efavirenz, Emtricitabine, and Tenofovir DF. The percentage is determined by dividing the total amount of Tenofovir DF plus the three degradation products into the amount of individual degradation product as determined by the HPLC assay.

The one component formulation of the present invention has the advantage that the different active pharmaceutical ingredients (APIs) do not have to be formulated as a two- or multicomponent system wherein direct physical contact between two of the APIs or between one of the APIs and one of the other compounds such as excipients, and in particular surfactants, such as is disclosed in US2007099902. The one component formulation can be easily formulated by combining Efavirenz and Tenofovir disoproxil hemifumarate, or alternatively by combining the three APIs in one and the same composition without the need for separating the different ingredients in different components.

Preferably, the APIs Tenofovir disoproxil hemifumarate, Emtricitabine and Efavirenz are formulated in an essentiality homogenous composition. This means that the three APIs are in direct contact with each other, i.e. without the presence of intermediate layers etc. The three APIs are preferably formulated in a one component formulation, optionally in combination with suitable excipients, for instance of the type as listed herein elsewhere.

In a preferred composition, the total amount of Efavirenz, Emtricitabine and Tenofovir disoproxil hemifumarate is greater than about 60 wt % drawn on the composition. Preferred compositions may further comprise magnesium stearate, croscarmellose sodium, microcrystalline cellulose and hydroxypropyl cellulose. The approximate percentages by weight of Efavirenz, Tenofovir hemifumarate, Emtricitabine, magnesium stearate, croscarmellose sodium, microcrystalline cellulose, and hydroxypropyl cellulose are, respectively, about 39, about 19, about 13, about 2, about 7, about 17, about 1 and about 2. These percentages may also be varied according to the needs of the formulation. In preferred embodiments, Efavirenz, Emtricitabine and Tenofovir disoproxil hemifumarate are provided (for the manufacture of a medicament) for the treatment of a (HIV) patient upon oral administration at substantially the same AUC and Cmax as the FDA approved products Truvada and Sustiva.

The composition of the present invention is preferably formulated by wet granulation.

Wet granulation is a process of using a liquid binder or adhesive to the powder mixture. The amount of liquid can be properly managed, and over wetting will cause the granules to be too hard and under wetting will cause them to be too soft and friable. Aqueous solutions have the advantage of being safer to deal with than solvents.

Wet granulation typically comprises weighing and blending the active ingredient(s) together with filler, disintegration agents etc. The wet granulate is prepared by adding the liquid binder/adhesive. Examples of binders/adhesives include aqueous preparations of cornstarch, natural gums such as acacia, cellulose derivatives such as methyl cellulose, CMC, gelatin, and povidone. Ingredients are placed within a granulator which helps ensure correct density of the composition. After the granules are dried, they are passed through a screen to select granules of uniform size to allow an even fill in the die cavity. Water mixed into the powder can form bonds between powder particles that are strong enough to lock them in together. However, once the water dries, the powders may fall apart and therefore might not be strong enough to create and hold a bond. Povidone also known as polyvinyl pyrrolidone (PVP) is one of the most commonly used pharmaceutical binders. PVP and a solvent are mixed with the powders to form a bond during the process, and the solvent evaporates. Once the solvent evaporates and powders have formed a densely held mass, then the granulation is milled which results in formation of granules. Wet granulation can be carried in a variety of equipment such as a rotary processor or a fluidised bed such as for instance described in Kristensen J, Hansen VW. Wet Granulation in Rotary Processor and Fluid Bed: Comparison of Granule and Tablet Properties. AAPS PharmSciTech. 2006; 7(1): Article 22.

Another aspect of the present invention relates to a method for the preparation of a composition comprising Tenofovir disoproxil hemifumarate, Efavirenz and Emtricitabine comprising a step of wet granulation of Tenofovir disoproxil hemifumarate, Efavirenz and Emtricitabine.

### BRIEF DESCRIPTION OF THE DRAWINGS

FIG. 1 HPLC spectrogram of Emtricitabine (Starting material);

FIG. 2 HPLC spectrogram of Tenofovir disoproxil fumarate (Starting material);

FIG. 3 HPLC spectrogram of Efavirenz (Starting material);

FIG. 4 HPLC spectrogram of Emtricitabine/Tenofovir disoproxil fumarate (30% w/w water);

FIG. 5 MS-Spectrum at min: 36.217 (Impurity);

FIG. 6 HPLC spectrogram of Emtricitabine/Tenofovir disoproxil fumarate (40% w/w water);

FIG. 7 MS-Spectrum at min: 26.573 (Impurity);

FIG. 8 MS-Spectrum at min: 36.129 (Impurity);

FIG. 9 HPLC spectrogram of Emtricitabine/Tenofovir disoproxil hemifumarate (30% w/w water);

FIG. 10 HPLC spectrogram of Emtricitabine/Tenofovir disoproxil hemifumarate (40% w/w water);

FIG. 11 HPLC spectrogram of Emtricitabine/Tenofovir disoproxil fumarate/Efavirenz (30% w/w water);

FIG. 12 MS-Spectrum at min: 26.573 (Impurity);

FIG. 13 MS-Spectrum at min: 36.129 (Impurity);

FIG. 14 HPLC spectrogram of Emtricitabine/Tenofovir disoproxil fumarate/Efavirenz (40% w/w water);

FIG. 15 MS-Spectrum at min: 26.573 (Impurity);

FIG. 16 MS-Spectrum at min: 36.129 (Impurity);

FIG. 17 HPLC spectrogram of Emtricitabine/Tenofovir disoproxil hemifumarate/Efavirenz (30% w/w water);

FIG. 18 HPLC spectrogram of Emtricitabine/Tenofovir disoproxil hemifumarate/Efavirenz (40% w/w water);

FIG. 19 HPLC chromatogram of Atripla sample prepared with 30% water after 33 weeks;

FIG. 20 HPLC chromatogram of Atripla sample prepared with 40% water after 33 weeks;

FIG. 21 HPLC chromatogram of Truvada sample prepared with 30% water after 33 weeks;

FIG. 22 HPLC chromatogram of Truvada sample prepared with 40% water after 33 weeks;

FIG. 23 HPLC chromatogram of Atripla sample prepared with 30% water after 33 weeks;

FIG. 24 HPLC chromatogram of Atripla sample prepared with 40% water after 33 weeks;

FIG. 25 HPLC chromatogram of Truvada sample prepared with 30% water after 33 weeks; and

FIG. 26 HPLC chromatogram of Truvada sample prepared with 40% water after 33 weeks.

### DETAILED DESCRIPTION OF THE INVENTION

### Examples

Analytical Methods: HPLC Assay for Degradation Product

Emtricitabine/Tenofovir disoproxil (Fumarate or Hemifumarate) granules are assayed by HPLC for Emtricitabine and Tenofovir disoproxil (Fumarate or Hemifumarate) using external references as described in US2007/0077295. The presence of degradation products are determined by area normalization. The identities of Emtricitabine and Tenofovir disoproxil (Fumarate or Hemifumarate) are confirmed by comparison of their retention times with those of the reference standards.

Standard and Sample Solvent: 25 Mm Phosphate Buffer pH 3

3.4 g of potassium phosphate monobasic, anhydrous is weighed and transferred into a 1 L volumetric flask. About 800 mL of water is added and mixed until dissolved. The pH to 3.0±0.1 is adjusted with phosphoric acid, then diluted to volume with water. Sample solvent (mixture of 25 mM phosphate buffer pH 3 40%: Acetonitrile 30%: methanol 30%): 400 mL of 25 mM phosphate buffer pH 3, 300 mL acetonitrile, 300 mL methanol is combined, mixed and allowed to equilibrate to ambient temperature. 50:50 Acetonitrile: methanol: Combine 500 mL acetonitrile and 500 mL methanol is combined, mixed and allowed to equilibrate to ambient temperature. Standard solution: 20 mg of Emtricitabine reference standard and 30 mg of Tenofovir disoproxil reference standard was weighed and transferred into a 100 mL volumetric flask. Approximately 80 mL of sample solvent was added (as prepared in step 2) to the flask and mixed or sonicated until dissolved. Diluted to volume with sample solvent (40:30:30) and mixed well. The final concentration of each component is approximately 0.2 mg/mL of Emtricitabine and 0.3 mg/mL Tenofovir disoproxil.

Sample Preparation for Emtricitabine/Tenofovir Disoproxil (Fumarate or Hemifumarate) Granules.

Approximately 6520 mg of Emtricitabine/Tenofovir disoproxil (Fumarate or Hemifumarate) granules was weighted into a 1 L volumetric flask. Added was 400 mL 25 mM phosphate buffer, pH 3 to the volumetric flask. Mixing was performed by stirring vigorously for about 75 minutes. 50:50 acetonitrile: methanol was added to the flask to approximately 2 cm below the 1 L mark. The solution was equilibrated to ambient temperature by mixing for 1 hour. The volume was diluted to 1 L with 50:50 acetonitrile: methanol and mixed well by stirring with a magnetic stirring bar. Using a 0.45 μm syringe filter with a syringe, approximately 10 mL for the next dilution was filtered. The first 2 mL of the filtrate was discarded. A class A pipette was used to transfer 5.0 mL of the filtrate into a 50 mL volumetric flask and dilute the to volume with sample solvent (40:30:30).

Chromatography

An LCMS system with UV detector, HP1100 API-ES MSD VL-type detector and electronic data acquisition system was used. An HPLC column, 4.6 mm i.d. by 100 mm long, packed with C18 reversed phase, 3.5 μm particle size 80 Å pore size material was used Mobile phase buffer: a 20 mM ammonium acetate buffer pH 4.6; adjust pH with acetic acid. Mobile phase gradient: mobile phase buffer: acetonitrile from 99:1 to 1:99 over 67 minutes. Peak detection: UV at 265 nm Injection volume 5 μL. Under the stated chromatographic conditions the retention times of Emtricitabine is 7.5 minutes. The retention time of he Tenofovir disoproxil is around 25 minutes.

HPLC Characterization of Starting Materials

The primary analytical method for HPLC characterization of starting material like Emtricitabine, Tenofovir disoproxil fumarate or hemifumarate, Efavirenz are described in herein elsewhere. This method was used to identify the purity of starting material.

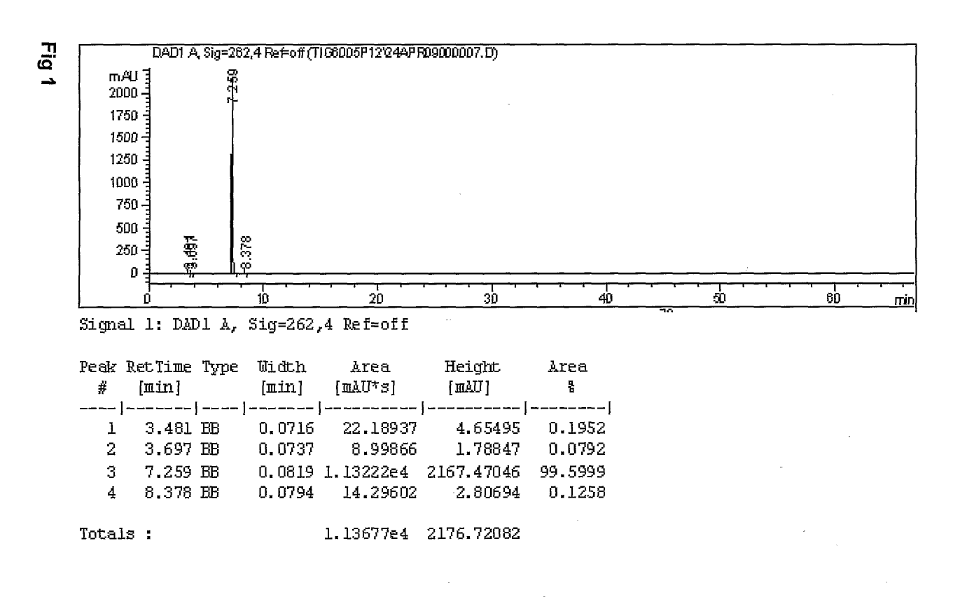

As shown in FIG. 1 the peak at retention time 7.259 min is for Emtricitabine and the peaks at 3.481 min, 3.697 min and 8.378 mins are the retentions peaks of Emtricitabine related compounds (impurities).

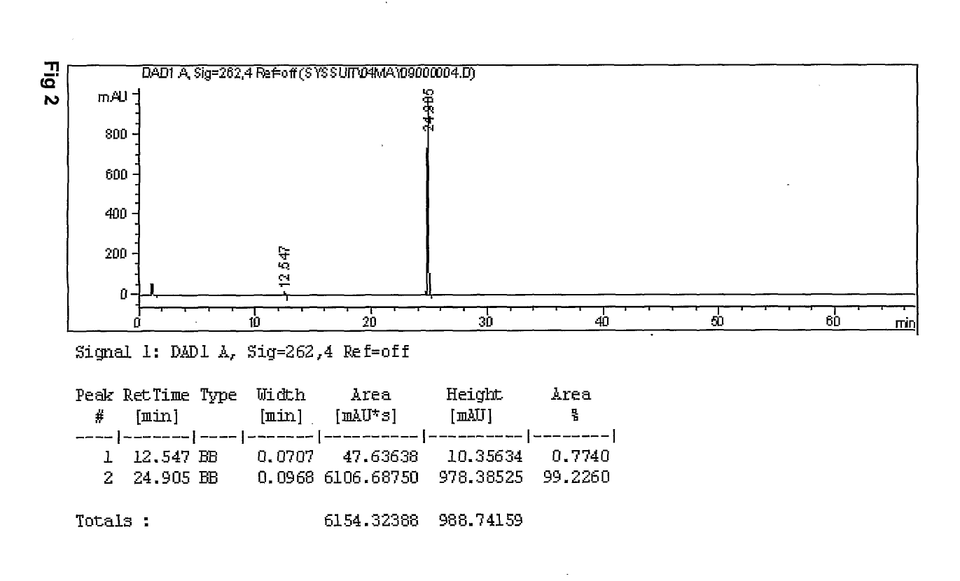

The FIG. 2 shows Tenofovir disoproxil fumarate peak at retention time 24.905 min. The FIG. 2 also shows the peak at retention time 12.547 min which corresponds to a TDF related product.

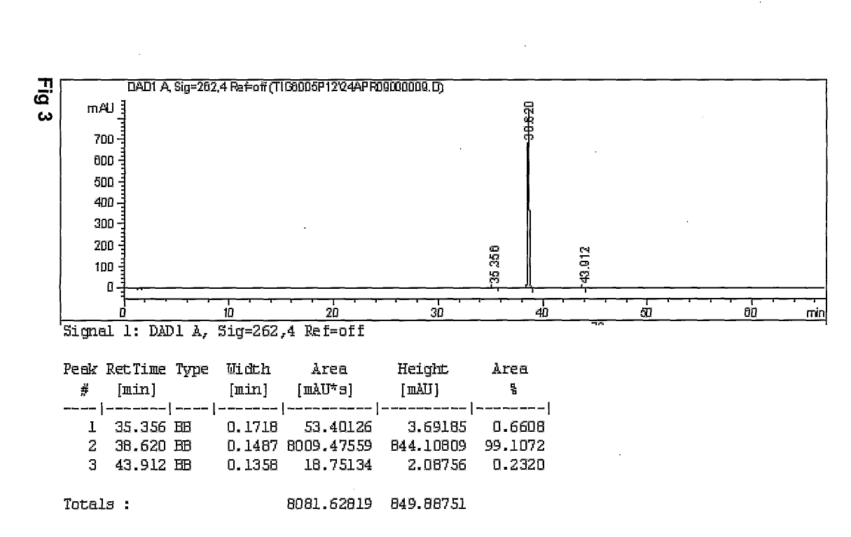

The Efavirenz HPLC chromatogram with a peak at retention time 38.620 min is shown in FIG. 3. The chromatogram also shows small peaks at retention time 35.356 min and 43.912 min which are Efavirenz related impurities.

Experimental procedure 1: Wet granulation according to US2007/0077295 of Emtricitabine with Tenofovir disoproxil Fumarate or Tenofovir disoproxil Hemifumarate.

A composition comprising the ingredients and ratios as listed in table 1 are subjected to a standard wet granulation process with respectively 30% and 40% w/w water. The quality of the granulation was assessed visually. The experiments were carried out by using a fluidized bed which was designed to carry out the wet granulation experiments of Tenofovir disoproxil fumarate or hemifumarate and Emtricitabine.

TABLE 1
Quantitative composition of Emtricitabine/Tenofovir
disoproxil Fumarate or Tenofovir disoproxil hemifumarate
for wet granulation experiment

| | % w/w of | Unit formula | Unit formula |
|---|---|---|---|
| Ingredient | total | (mg/tablet) | (mg/tablet) |
| Emtricitabine | 30.68 | 200 | 200 |
| Tenofovir disoproxil Fumarate | 46.00 | | 300 |
| Tenofovir disoproxil | 46.00 | 300 | |
| Hemifumarate | | | |
| Micro crystalline cellulose | 13.72 | 89.5 | 89.5 |
| Croscarmellose sodium | 7.37 | 48 | 48 |
| Magnesium stearate | 2.23 | 14.5 | 14.5 |

Experimental procedure 2: Wet granulation of Emtricitabine, Tenofovir disoproxil fumarate or Hemifumarate and Efavirenz.

Combinations of Tenofovir disoproxil fumarate with Emtricitabine/Efavirenz or Tenofovir disoproxil hemifumarate with Emtricitabine/Efavirenz were formulated in order to investigate the quality of the resulting granules of the combination. The wet granulation experiment of Tenofovir disoproxil fumarate with Emtricitabine and Efavirenz or Tenofovir disoproxil hemifumarate with Emtricitabine and Efavirenz similar to the Atripla formulation with reduced amounts of excipients were carried out to check the chemical stability of Tenofovir disoproxil hemifumarate co-crystals.

A composition comprising the ingredients and ratios as listed in table 2 were subjected to a standard wet granulation process with respectively 30% and 40% w/w water. The quality of the granulation was assessed visually. The experiments were carried out by using fluidized bed which was designed to carry out the wet granulation experiments of Tenofovir disoproxil fumarate or hemifumarate, Emtricitabine and Efavirenz.

TABLE 2
Quantitative composition of Emtricitabine/(Tenofovir
disoproxil Fumarate or Tenofovir disoproxil hemifumarate)/Efavirenz
for wet granulation experiment

| | % w/w of | Unit formula | Unit formula |
|---|---|---|---|
| Ingredient | total | (mg/tablet) | (mg/tablet) |
| Emtricitabine | 13.07 | 400 | 400 |
| Tenofovir disoproxil | 19.59 | 600 | |
| Fumarate | | | |
| Tenofovir disoproxil | 19.59 | | 600 |
| Hemifumarate | | | |
| Efavirenz | 39.17 | 1201.3 | 1201.3 |
| Micro crystalline | 17.08 | 523.3 | 523.3 |
| cellulose | | | |
| Croscarmellose sodium | 6.27 | 192 | 192 |
| Magnesium stearate | 1.58 | 48.4 | 48.4 |
| Hydroxy propyl cellulose | 2.53 | 76.8 | 76.8 |
| Sodium lauryl sulphate | 0.78 | 24 | 24 |

Wet Granulation of Tenofovir Disoproxil Fumarate with Emtricitabine (by Using 30% w/w High Pure Water)

The wet granulation of Emtricitabine/Tenofovir was carried out with the composition mentioned in Table 1 by using 30% w/w high pure water. The evaluation was done by integrating the retention peaks of LCMS spectrum which was obtained during the analysis as shown in FIG. 1. The analytical method as mentioned above was used to determine the concentration of Emtricitabine/Tenofovir disoproxil fumarate.

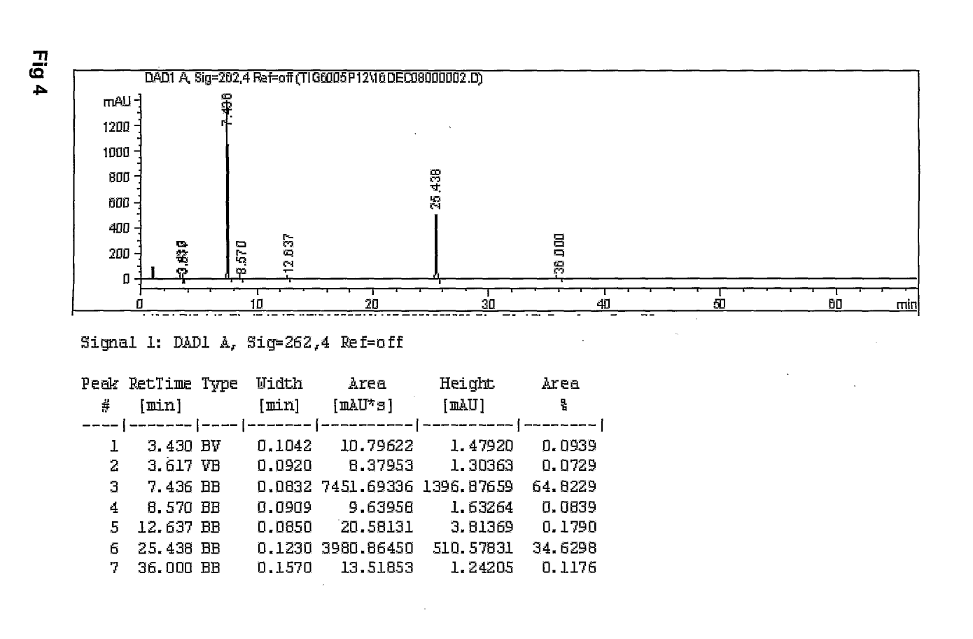

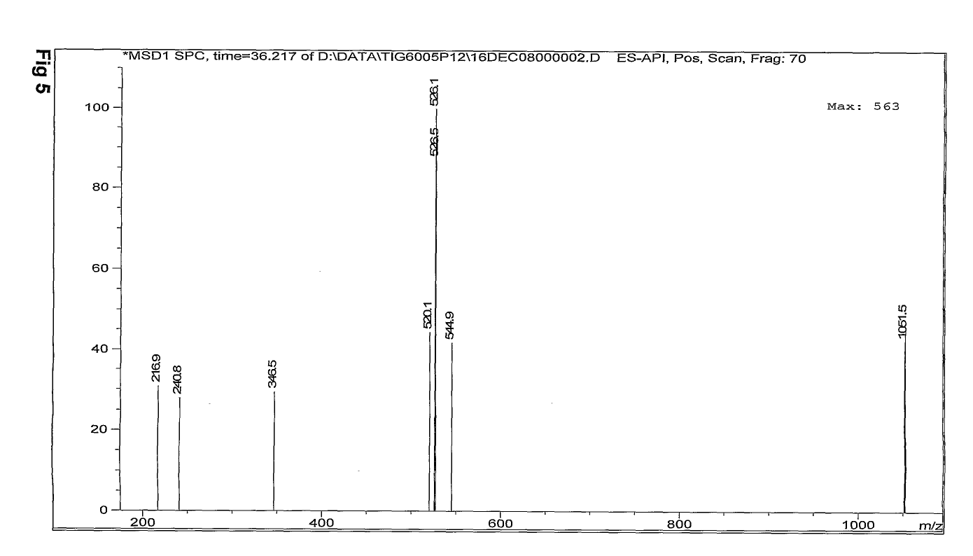

The retention time 7.45 min as shown in FIG. 4 corresponds to Emtricitabine and 3.62, 8.57, 12.63 min are also corresponds to Emtricitabine related products. The 25.438 min corresponds to Tenofovir disoproxil fumarate retention during the HPLC analysis. The retention peak at 36.00 min shown in FIG. 4 which has a mass of 1051.5 shown in the mass spectra in FIG. 5.

Wet Granulation of Tenofovir Disoproxil Fumarate with Emtricitabine (by Using 40% w/w High Pure Water)

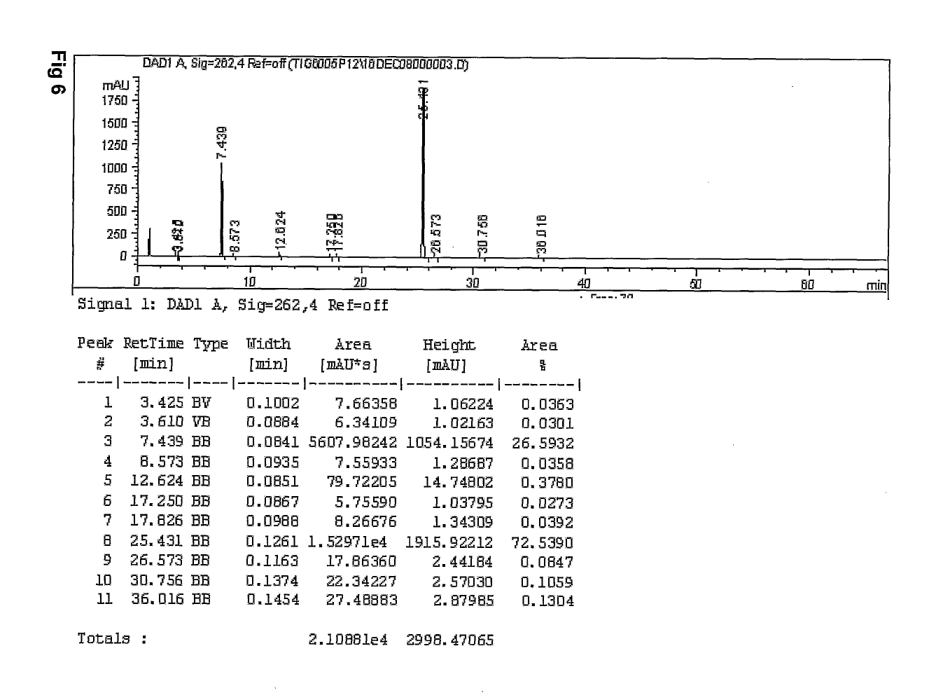

The wet granulation experiment of Emtricitabine and Tenofovir disoproxil fumarate as described in Table 1 was carried out using 40% w/w high pure water. The purity analysis was conducted by HPLC as mentioned above. FIG. 6 shows the HPLC spectrogram containing Emtricitabine retention peak at 7.439 min and Tenofovir disoproxil fumarate retention peak at 25.431 min.

FIG. 6 shows the HPLC spectrogram with the retention peaks at 3.425, 3.610, 8.573 and 12.624 min which corresponds to the Emtricitabine related compounds.

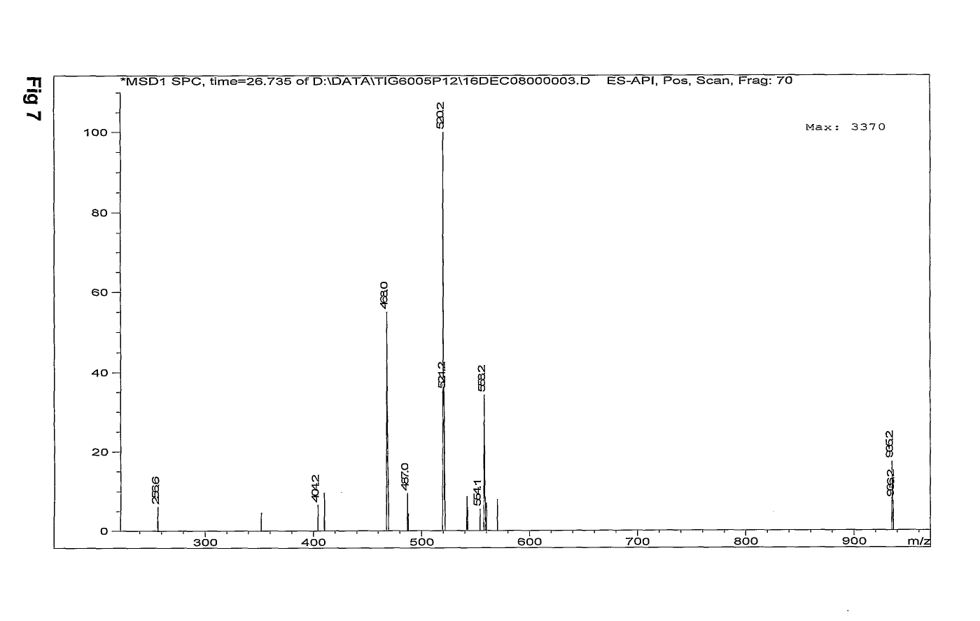

As shown in FIG. 7 the mass spectra with mass 935.2 was obtained by integrating the HPLC peak at 26.573 min. FIG. 8 also shows the mass spectra of the peak at 36 min. The mass of the product is 1051.4.

Wet Granulation experiment on Tenofovir disoproxil hemifumarate with Emtricitabine.

The wet granulation experiments of Tenofovir disoproxil hemifumarate and Emtricitabine suitable for Atripla formulation or Truvada formulation with reduced amounts of excipients were carried out to check the chemical stability of Tenofovir disoproxil hemifumarate

A composition comprising the ingredients and ratios as listed in Table 1 are subjected to a standard wet granulation process with respectively 30% and 40% w/w water. The quality of the granulation was assessed visually. The experiments were carried out by using fluidized bed.

Wet Granulation of Tenofovir Disoproxil Hemifumarate with Emtricitabine by Using 30% w/w High Pure Water.

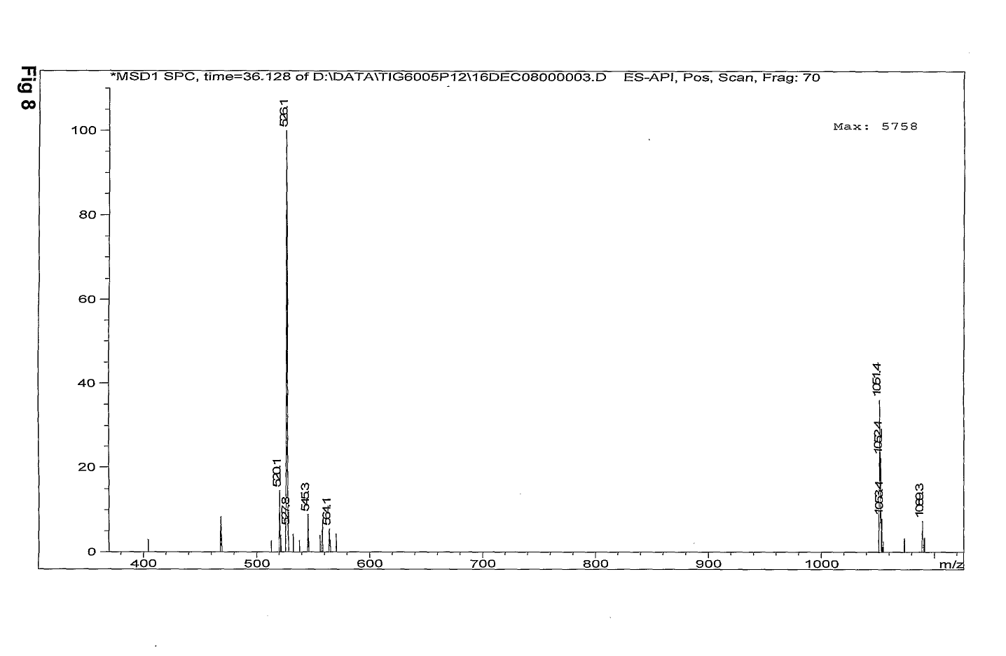

The wet granulation experiment of Emtricitabine and Tenofovir disoproxil hemifumarate was carried out as described above for the fumarate by using 30% w/w high pure water. FIG. 9 shows the HPLC spectra of Tenofovir disoproxil hemifumarate at retention time 25.328 min and the peak of Emtricitabine at 7.378 min. The peaks at retention time 3.544, 3.766 and 8.536 are Emtricitabine related compounds. The impurity peaks of Tenofovir disoproxil at 26.57 and 36.0 were not observed during the wet granulation experiments with Tenofovir disoproxil hemifumarate.

Wet Granulation of Tenofovir Disoproxil Hemifumarate with Emtricitabine by Using 40% w/w High Pure Water.

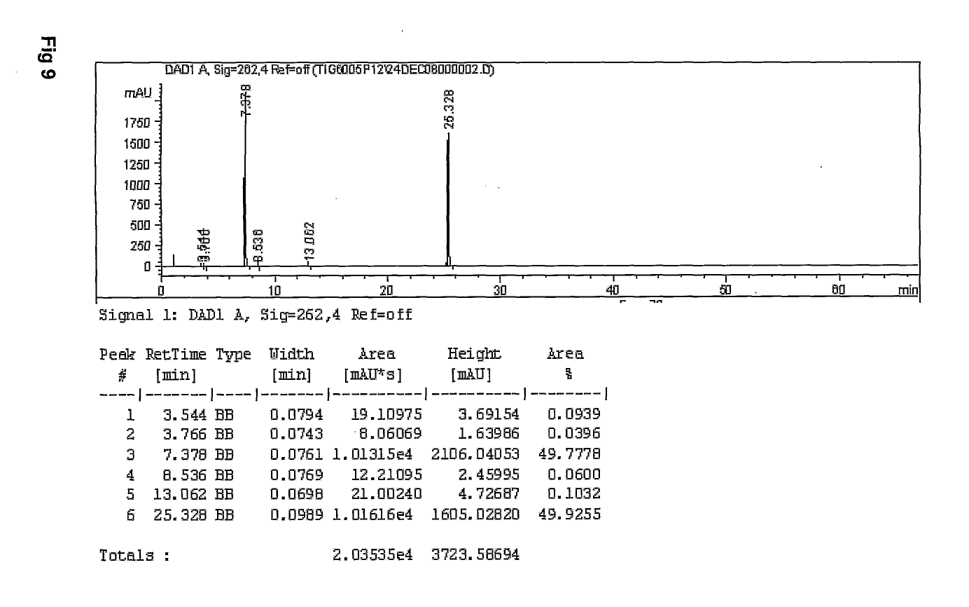

The wet granulation experiment of Emtricitabine and Tenofovir disoproxil hemifumarate as described above was carried out by using 40% w/w high pure water. FIG. 10 is the HPLC spectrogram of Tenofovir disoproxil hemifumarate peak at retention time 25.425 min and the peak of Emtricitabine at 7.441 min. The peak at retention time 13.062 min also relates to the Emtricitabine related product. The impurity peaks of Tenofovir disoproxil at 26.57 and 36.0 were not observed during the wet granulation experiments with Tenofovir disoproxil hemifumarate.

A wet granulation technique can be successfully employed to manufacture Emtricitabine/Tenofovir disoproxil hemifumarate tablets without forming degradation products of Tenofovir disoproxil.

Wet Granulation of Tenofovir Disoproxil Fumarate with Emtricitabine and Efavirenz (30% w/w High Pure Water)

The Atripla formulation of Tenofovir disoproxil fumarate, Emtricitabine and Efavirenz by using 30% w/w high pure water was carried out to identify the stability of Tenofovir disoproxil fumarate in the presence of SLS in line with US2007099902.

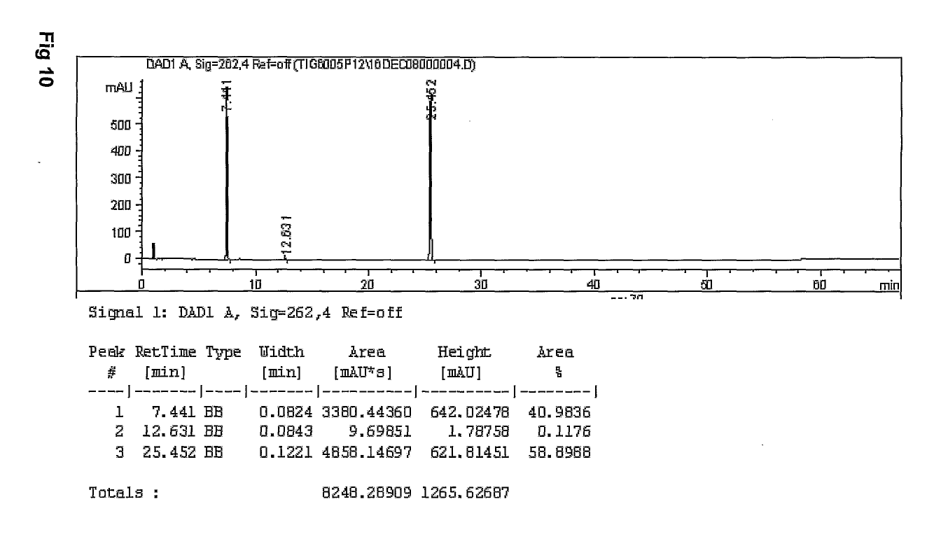

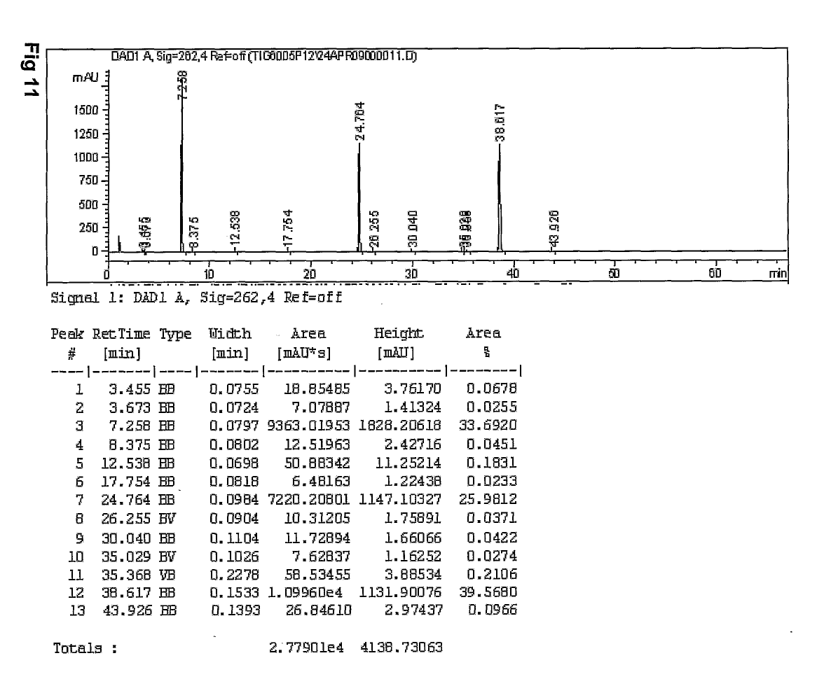

FIG. 11 shows the HPLC chromatogram with major peaks at retention time 7.258 min, 24.764 min and 38.617 min which corresponds to Emtricitabine, Tenofovir disoproxil fumarate and Efavirenz respectively. After integrating the peaks at retention time 26.255 min and 35.029 min on a mass spectrometer which shows a mass of 935.2 and 1051.4 respectively which corresponds to the impurities mentioned in the US2007099902. The mass spectrograms of impurities are shown in FIG. 12 and FIG. 13.

Wet Granulation of Tenofovir Disoproxil Fumarate with Emtricitabine and Efavirenz (40% w/w High Pure Water)

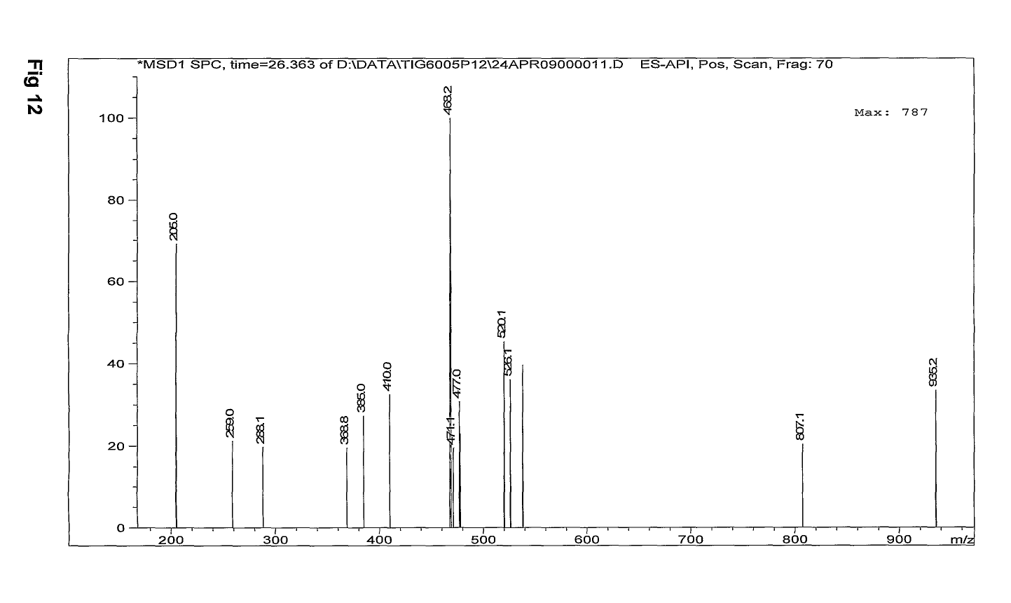

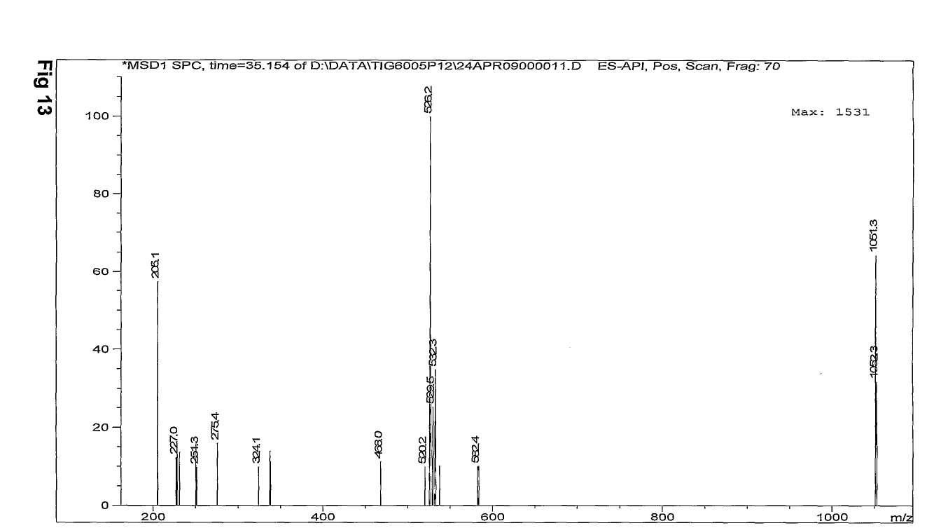

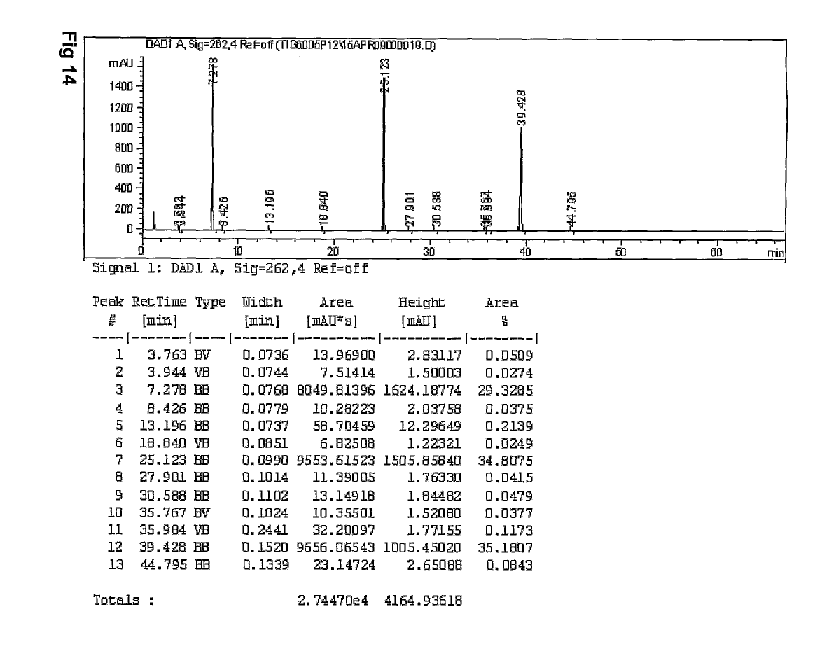

FIG. 14 shows the HPLC Chromatogram of the final product obtained by wet granulating Emtricitabine, Tenofovir disoproxil fumarate and Efavirenz as mentioned in table 2. The retention peaks at 7.278 min, 25.123 min and 39.428 min are the retention peaks of Emtricitabine, Tenofovir disoproxil fumarate and Efavirenz respectively. The figure also indicates the small retention peaks at 27.901 min, 30.588 min and 35.767 min corresponds to the Tenofovir disoproxil fumarate impurities which were obtained during the wet granulation experiments. FIG. 15 and FIG. 16 shows the mass spectra of impurities of mass 935.2 and 1051.4 which belong to the retention peaks 27.901 min and 35.767 min.

Wet Granulation of Tenofovir Disoproxil Hemifumarate with Emtricitabine and Efavirenz (30% w/w High Pure Water)

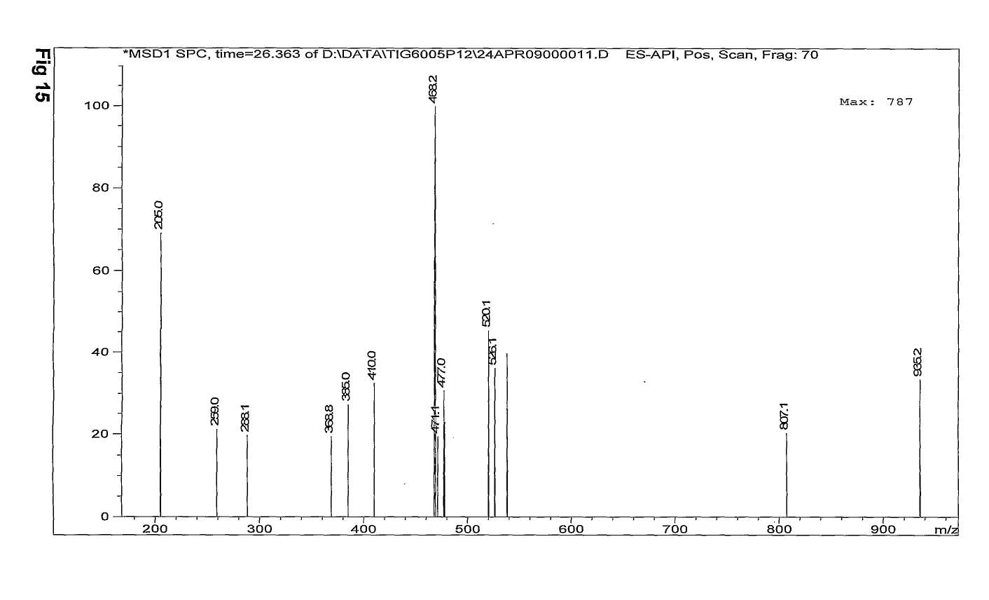

As described in the table 2 the same composition was used to formulate the Emtricitabine, Tenofovir disoproxil hemifumarate and Efavirenz. FIG. 17 shows the retention peaks at 7.356 min, 25.388 min and 39.402 min are Emtricitabine, Tenofovir disoproxil hemifumarate and Efavirenz respectively. It is clear from FIG. 17 that the wet granulation technique can be successfully employed to manufacture Emtricitabine/Tenofovir disoproxil hemifumarate/Efavirenz tablets (Atripla formulation) without forming degradation products of Tenofovir disoproxil.

Wet Granulation of Tenofovir Disoproxil Hemifumarate with Emtricitabine and Efavirenz (40% w/w High Pure Water)

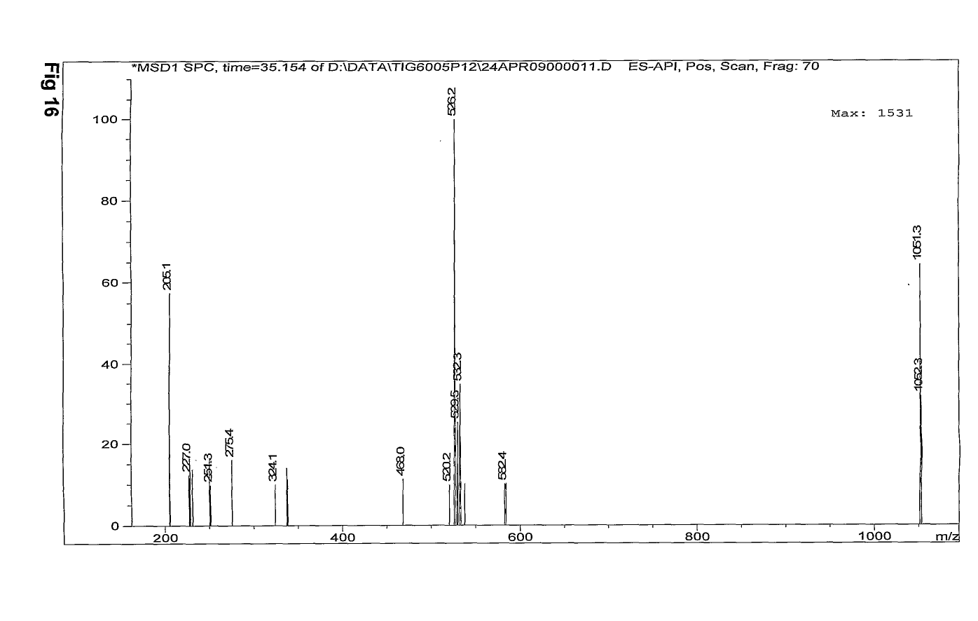

Wet granulation of Emtricitabine/Tenofovir disoproxil hemifumarate/Efavirenz was carried out using 40% w/w high pure water to identify the stability of the combination product. FIG. 18 shows the HPLC chromatogram with major peaks at retention time 7.373 min, 25.375 min and 39.481 min which corresponds to Emtricitabine, Tenofovir disoproxil hemifumarate and Efavirenz respectively. The peaks at retention time 4.053 min, 4.183 min and 8.509 are the Emtricitabine related impurities. The minor peaks at 35.981 min and 44.838 min are the same impurities observed during the starting material characterization of Efavirenz which is also shown in FIG. 3.

From the above studies it can be concluded that the co-crystalline form of Tenofovir disoproxil hemifumarate is stable during the wet granulation of Tenofovir disoproxil hemifumarate with Emtricitabine and of Tenofovir disoproxil hemifumarate with, Emtricitabine and Efavirenz following Truvada (table 1) and Atripla (table 2) formulations. The co-crystalline form of Tenofovir disoproxil hemifumarate provides the good chemical stability for Emtricitabine/Tenofovir/Efavirenz formulations and it is preferred over the Tenofovir disoproxil fumarate (1:1).

Stability Testing at 33 Weeks

The stability of the Truvada and Atripla combination formulations after wet granulation was checked by HPLC after 33 weeks storage under 75% Relative Humidity (RH) and 40° C. The results are shown in the sections below.

Two types of formulations were prepared for each combination, one containing the Tenofovir Disoproxil Fumarate and the other one containing the Tenofovir Disoproxil Hemifumarate co-crystal. In both combinations, water content of 30 and 40% was prepared.

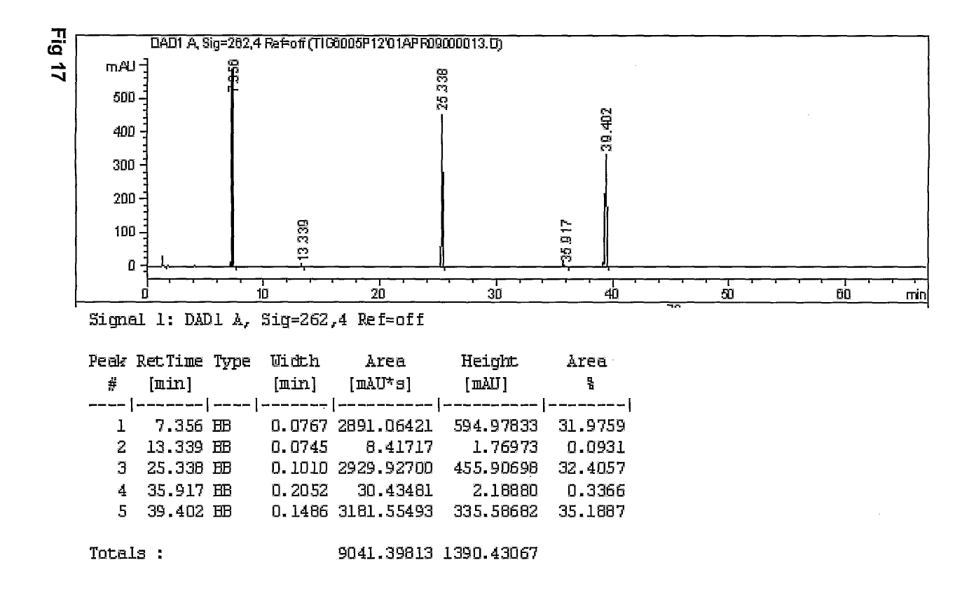

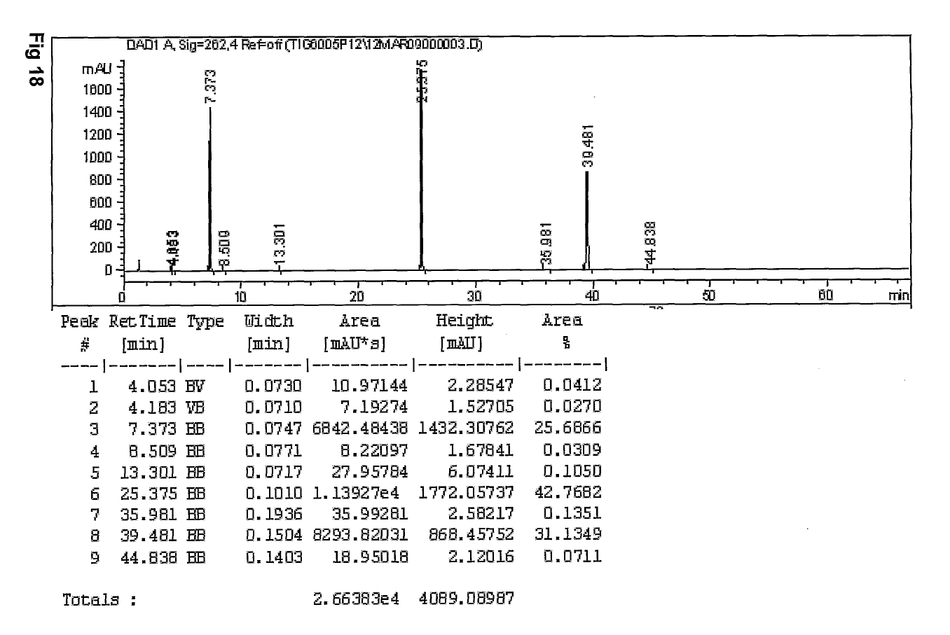

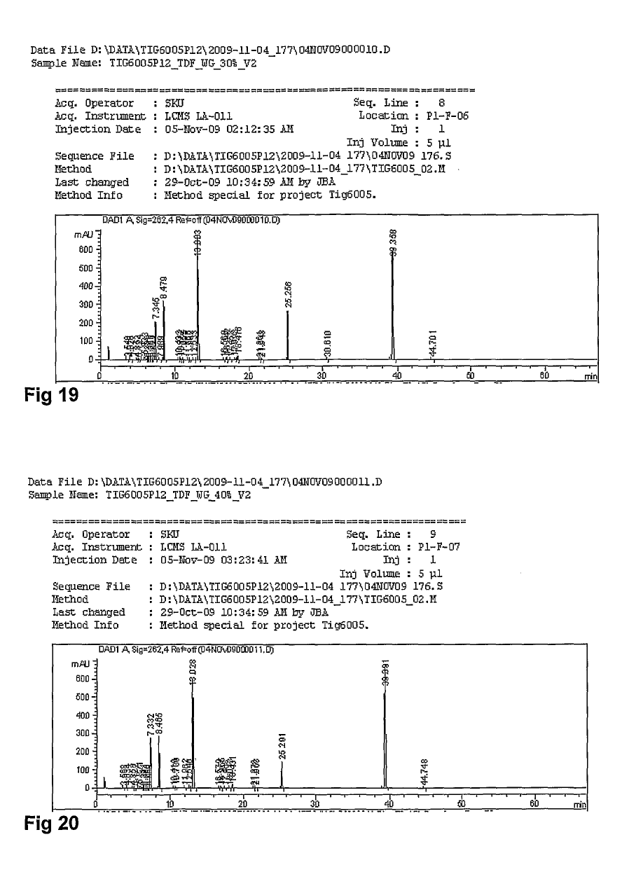

The HPLC chromatogram of Atripla sample prepared with 30% water shows a degradation of Tenofovir disoproxil fumarate to a large extent (FIG. 19).

The relatively high impurity peaks at retention times 13.00 min with a mass of 405.1 and at 8.48 minutes, as well as the relatively lower impurity peaks at retention times 30.61, 16.59, 16.90, 17.41, 17.96, 18.30 minutes are all related to Tenofovir disoproxil.

The HPLC chromatogram of Atripla sample prepared with 40% water shows a degradation of Tenofovir disoproxil to a large extent (FIG. 20).

The relatively high impurity peaks at retention times 13.00 min with a mass of 405.1 and at 8.48 minutes, as well as the relatively lower impurity peaks at retention times 16.57, 16.95, 17.46, 18.01, 18.43, 21.37 and 21.65 minutes are all related to Tenofovir disoproxil.

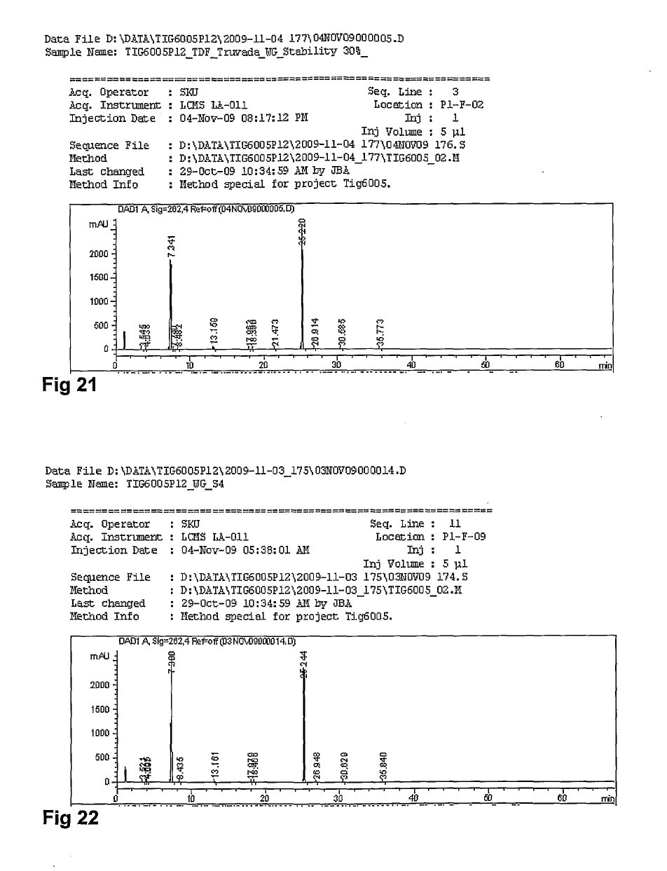

The HPLC chromatogram of Truvada sample prepared with 30% water shows a degradation of Tenofovir disoproxil fumarate to a large extent (FIG. 21).

The impurity peaks at retention times 13.15, 17.90, 18.3, 21.47, 26.91, 30.58, 35.77 minutes are all related to Tenofovir disoproxil. The peaks at retention 26.91, 30.58 and 35.77 minutes are the impurities with a mass of 935, 606 and 1051.1 respectively.

The HPLC chromatogram of Truvada sample prepared with 40% water shows a degradation of Tenofovir disoproxil fumarate to a large extent (FIG. 22).

The impurity peaks at retention times 26.94, 30.629 and 35.84 minutes correspond to masses of 935, 606 and 1051.1 respectively.

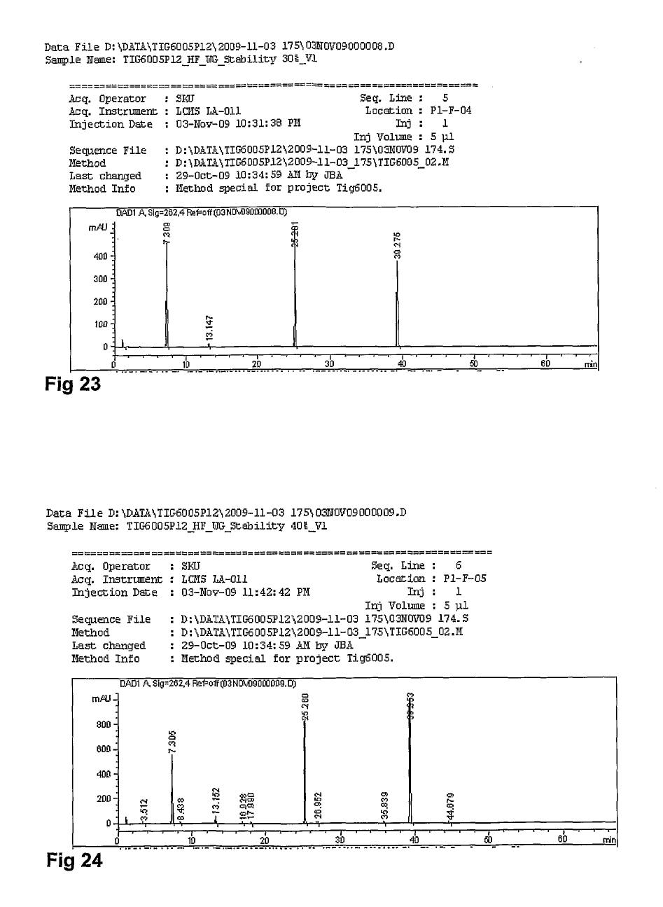

The HPLC chromatogram of Atripla sample prepared with 30% water shows no degradation of Tenofovir disoproxil hemi-fumarate (FIG. 23).

The HPLC chromatogram of Atripla sample prepared with 40% water (FIG. 6) shows less degradation of Tenofovir disoproxil hemi-fumarate compared to that of the corresponding formulation of Tenofovir disoproxil fumarate (FIG. 24).

The small peaks at retention times of 26.95 and 35.83 minutes are related to Tenofovir disoproxil and have masses of 935 and 1051.1 respectively.

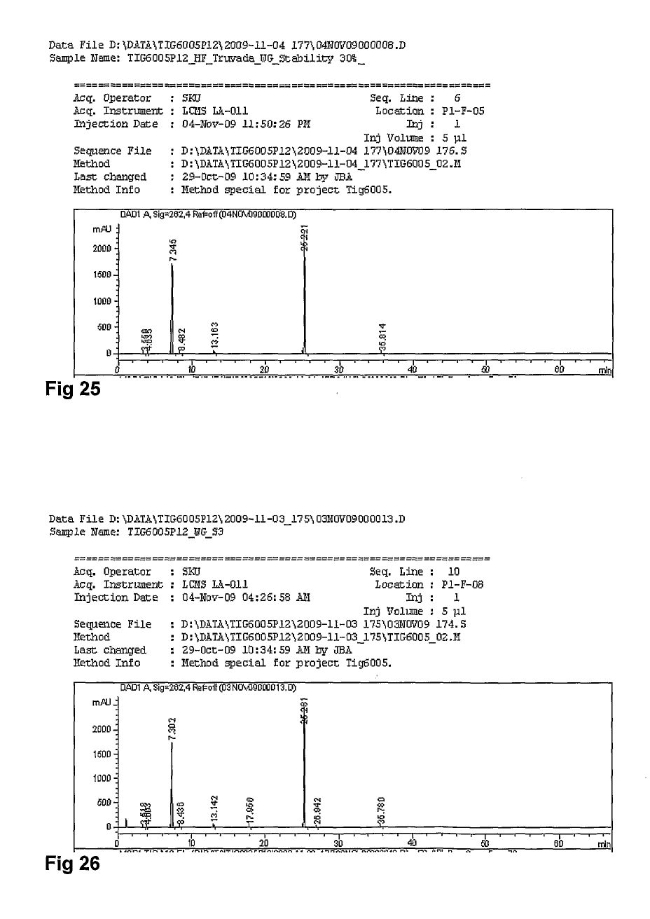

The HPLC chromatogram of Truvada sample prepared with 30% water shows a small degradation of Tenofovir disoproxil hemi-fumarate (FIG. 25).

The peak at retention time 35.81 minutes shows the Tenofovir disoproxil related impurity with a mass of 1051.1

The HPLC chromatogram of Truvada sample prepared with 40% water shows a small degradation of Tenofovir disoproxil hemi-fumarate (FIG. 26).

The peaks at retention time of 26.94 and 35.78 minutes are the Tenofovir disoproxil related impurities with a mass of 935 and 1051.1 respectively.

1.-13. (canceled)

14. A composition comprising Tenofovir disoproxil hemifumarate, Efavirenz and Emtricitabine wherein Tenofovir disoproxil hemifumarate, Efavirenz and Emtricitabine are in an essentially homogenous composition.

15. A composition according to claim 14, wherein the Tenofovir disoproxil hemifumarate, Efavirenz and Emtricitabine are in direct contact with each other.

16. A composition, according to claim 14, wherein the Tenofovir disoproxil hemifumarate, Efavirenz and Emtricitabine are in a one component formulation

17. A composition according to claim 14, produced by wet granulation of Tenofovir disoproxil hemifumarate, Efavirenz and Emtricitabine.

18. The composition of claim 14, wherein the total amount of Efavirenz, Emtricitabine and Tenofovir DF is greater than about 50%, preferably 60 wt % of the composition.

19. The composition of claim 14, which further comprises magnesium stearate, croscarmellose sodium, microcrystalline cellulose and hydroxypropyl cellulose.

20. The composition of claim 14, wherein the approximate percentages by weight of Efavirenz, Tenofovir DF, Emtricitabine, magnesium stearate, croscarmellose sodium, microcrystalline cellulose, sodium lauryl sulfate, and hydroxypropyl cellulose are, respectively, about 39, about 19, about 13, about 2, about 7, about 17, about 1 and about 2.

21. The composition of claim 14, wherein Efavirenz, Emtricitabine and Tenofovir DF are provided to a patient upon oral administration at substantially the same AUC and Cmax as the FDA approved products Truvada and Sustiva.

22. Method for the preparation of an essentially homogenous composition comprising Tenofovir disoproxil hemifumarate, Efavirenz and Emtricitabine comprising a step of wet granulation of Tenofovir disoproxil hemifumarate, Efavirenz and Emtricitabine.

23. Dosage form suitable for oral administration comprising Tenofovir disoproxil hemifumarate, Efavirenz and Emtricitabine in an essentially homogenous distribution.
